(19) United States
(12) Patent Application Publication
(10) Pub. No.: US 2012/0088698 A1
(43) Pub. Date: Apr. 12, 2012

(54) WATER IN OIL EMULSION, METHOD FOR THE PRODUCTION THEREOF

(75) Inventors: Christoph Kayser, Mainz (DE); Alexander Roesch, Oppenheim (DE); Gernold Botthof, Antrifttal (DE); Dirk Leinweber, Kelkheim (DE); Rainer Kupfer, Hattersheim (DE); Claudia Diemel, Gelnhausen (DE)

(73) Assignee: CLARIANT FINANCE (BVI) LIMITED, Tortola (VG)

(21) Appl. No.: 13/376,416

(22) PCT Filed: May 19, 2010
(86) PCT No.: PCT/EP2010/003062

§ 371 Date: Dec. 6, 2011

(30) Foreign Application Priority Data
Jun. 25, 2009 (DE) 10 2009 030 411.8

(51) Int. Cl. C09K 8/12; C07D 207/277

(52) U.S. Cl. 507/130; 548/531

(57) ABSTRACT

The invention relates to inverse emulsions, comprising a) a hydrophobic liquid as a continuous phase, b) water as a disperse phase, and c) a compound of the formula (1),

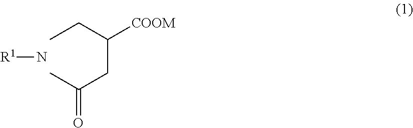

where R¹ is a hydrocarbon group having between 6 and 30 C atoms or a group R⁵—O—X-M hydrogen, alkali metal, alkali earth metal, or an ammonia group, and where R⁵ is a hydrocarbon group having between 6 and 30 carbon atoms, X is C₂-C₆-alkylene or a poly(oxyalkylene) group of the formula,

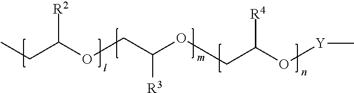

where l is a number between 1 and 50, m and n are numbers independent of l and of each other between 0 and 50, and R², R³, R⁴ are independent of each other and is hydrogen, CH₃, or CH₂CH₃, and Y is C₂-C₆-alkylene.

The present invention relates to a water-in-oil emulsion (hereinafter W/O emulsion or inverse emulsion) and to a process for production thereof, wherein substituted pyrrolidonecarboxylic acids are used as an emulsifier.

An emulsion is a dispersed mixture of two or more immiscible liquids, one of which is present dispersed in the other. In a conventional emulsion composed of water and oil, either the oil may be dispersed in the water (oil-in-water or O/W emulsion) or the water may be dispersed in the oil (water-in-oil W/O or inverse emulsion).

Emulsions are used in a multitude of fields, such as textile, leather and metal treatment, foods, cosmetics, pharmaceuticals, coating materials, in agrochemicals, in polymerization, in cleaning and polishing, and in ore extraction and natural gas and mineral oil production.

Emulsions are intrinsically unstable systems and the risk of deterioration in the properties thereof (for example as a result of emulsion splitting) during storage is greater than in the case a nonemulsified product. However, the sensible selection of the constituents thereof and a sensible production process can result in emulsions whose properties change only imperceptibly in the course of storage and use. Such emulsions fulfill important tasks in the abovementioned fields of use. The possible uses are extremely varied and range from foods such as mayonnaise to functional liquids, for example inverse drilling mud emulsions.

Important properties, for emulsions are the dilutability, viscosity, color and stability thereof. These properties depend on the chemical nature of the continuous phase and disperse phase, the ratio of the continuous to the disperse phase and the particle size of the disperse phase. In a particular emulsion, the properties depend on which liquid forms the continuous phase, i.e. whether the emulsion is O/W or W/O. The resulting emulsion is determined by the emulsifier (type and amount), the ratio of the ingredients and the sequence of addition of ingredients during the mixing.

The dispersibility (solubility) of the emulsion is determined by the continuous phase. Thus, if the continuous phase is water-soluble, the emulsion can be diluted with water. If, conversely, the continuous phase is oil-soluble, the emulsion can be diluted with oil.

An emulsion is stable provided that the particles of the disperse phase do not coalesce. The stability of an emulsion depends on the particle size, the difference in the density of the two phases, the rheological properties of the continuous phase and of the completed emulsion, the charges on the particles, the nature, efficacy and amount of the emulsifier used, the storage conditions, including temperature variation, movement and vibration or shaking, and dilution or evaporation during storage or use. The stability of an emulsion is influenced by virtually all factors involved in the formulation and preparation thereof. In the case of formulations containing large amounts of emulsifier, the stability is predominantly a function of the type and of the concentration of the emulsifier.

Emulsifiers can be classified as ionic or nonionic according to their characteristics. An ionic emulsifier is formed from an organic lipophilic group (L) and a hydrophilic group (H). The hydrophilic-lipophilic balance (FIB) is frequently used to characterize emulsifiers and related surfactant materials. The ionic types can be divided further into anionic and cationic, according to the nature of the ion-active group. The lipophilic component of the molecule is generally considered to be the surface-active component.

Nonionic emulsifiers are fully covalent and do not exhibit any obvious tendency to ionization. They can therefore be combined with other nonionic surfactants and likewise either with anionic or cationic substances. The nonionic emulsifiers are likewise less receptive to the effect of electrolytes than the anionic surfactants. The solubility of an emulsifier is of utmost significance in the preparation of emulsifiable concentrates.

DE-A-10 2007 015757 discloses the use of polyvinylpyrrolidones as a stabilizer for emulsions.

It was an object of the present invention to find emulsifiers for the production of inverse emulsions, which exhibit improved efficacy and improved biodegradability compared to the prior art emulsifiers.

It has been found that, surprisingly, substituted pyrrolidonecarboxylic acids and salts thereof are excellent emulsifiers for inverse emulsions.

The invention therefore provides inverse emulsions comprising
a) a hydrophobic liquid as a continuous phase
b) water as a disperse phase, and
c) a compound of the formula (1)

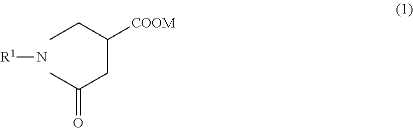

in which
R¹ is a hydrocarbyl group having 6 to 30 carbon atoms or an R⁵—O—X— group
M is hydrogen, alkali metal, alkaline earth metal or an ammonium group
R⁵ is a hydrocarbyl group having 6 to 30 carbon atoms
X is C₂-C₆-alkylene or a poly(oxyalkylene) group of the formula

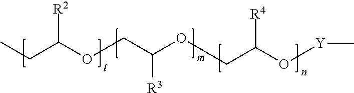

in which
l is a number from 1 to 50,
m, n are independent of l and are each independently a number from 0 to 50,
R², R³, R⁴ are each independently hydrogen, CH₃ or CH₂CH₃
Y is C₂-C₆-alkylene.

The invention further provides a process for producing an inverse emulsion, by adding a compound of the formula (1) to a mixture of a hydrophobic liquid and water.

The invention further provides for the use of a compound of the formula (1) as an emulsifier in inverse emulsions which comprise a hydrophobic liquid as a continuous phase and water as a disperse phase.

The compound of the formula (1) is also referred to hereinafter as inventive emulsifier.

In one embodiment, R¹ is a hydrocarbyl group, in which case R¹ does not contain any heteroatoms. R¹ is preferably C₈-C₃₀-alkyl, C₈-C₃₀-alkenyl, C₆-C₃₀-aryl or C₇-C₃₀-alkylaryl. More preferably, R¹ is a linear or branched C₈-C₂₄-alkyl or alkenyl chain, e.g. n- or isooctyl, n- or isononyl, or isodecyl, undecyl, dodecyl, tetradecyl, hexadecyl, octadecyl, eicosyl or longer radicals. Particular preference is given to cocoyl and oleyl radicals, R¹ may likewise be a C₆-C₃₀-aryl radical which is mono- or polycyclic and which may bear substituents, especially alkyl and/or alkenyl radicals. Additionally preferably, R¹ is a linear or branched, aliphatic C₁₂-C₂₄ hydrocarbyl radical having one or more double bonds.

R⁶ is preferably C₅-C₃₀-alkyl, C₈-C₃₀-alkenyl, C₆-C₃₀-aryl or C₇-C₃₀-alkylaryl. More preferably, R⁵ is a linear or branched C₈-C₂₄-alkyl or alkenyl chain, e.g. n- or isooctyl, n- or isononyl, n- or isodecyl, undecyl, tetradecyl, hexadecyl, octadecyl, eicosyl or longer radicals. Particular preference is given to cocoyl and oleyl radicals. R⁵ may likewise be a C₆-C₃₀-aryl radical which is mono- or polycyclic and which may bear substituents, especially alkyl and/or alkenyl radicals. Additionally preferably, R⁶ is a linear or branched, aliphatic C₁₂-C₂₄ hydrocarbyl radical having one or more double bonds.

X and Y are preferably each a group of the formula —(CHR¹⁶)ₖ— in which R¹⁶ is H, CH₃ or CH₂CH₃ and k is a number from 2 to 6. R¹⁶ is preferably H. k is preferably a number from 2 to 4. More preferably, —(CHR¹⁸)ₖ— represents groups of the formulae —CH₂—CH₂—, —CH₂—CH(CH₃)—, —(CH₂)₃— or —CH₂—CH(CH₂CH₃)—, R¹⁶ may have the same definition in all —(CH₂R¹⁶)— units, or different definitions.

l is preferably a number from 2 to 10.

m is preferably a number from 1 to 10. In a further preferred embodiment, m is zero, 1, 2 or 3

n is preferably a number from 1 to 10. In a further preferred embodiment, m is zero, 1, 2 or 3 and n is zero.

The pyrrolidonecarboxylic acids of the formula (1), when M is H, can be converted to salts by neutralization.

Suitable neutralizing agents are amines of the formula (2)

NR⁷R⁸R⁹ (2)

in which R⁷, R⁸ and R⁹ are each independently hydrogen or a hydrocarbyl radical having 1 to 100 carbon atoms.

In a first preferred embodiment, R⁷ and/or R⁸ and/or R⁹ are each independently an aliphatic radical. This has preferably 1 to 24, more preferably 2 to 18 and especially 3 to 6 carbon atoms. The aliphatic radical may be linear, branched or cyclic. It may additionally be saturated or unsaturated. The aliphatic radical is preferably saturated. The aliphatic radical may bear substituents, for example hydroxyl, C₁-C₅-alkoxy, cyano, nitrile, nitro and/or C₅-C₂₀-aryl groups, for example phenyl radicals. The C₅-C₂₀-aryl radicals may themselves optionally be substituted by halogen atoms, halogenated alkyl radicals, C₁-C₂₀-alkyl, C₂-C₂₀-alkenyl, hydroxyl, C₁-C₆-alkoxy, for example methoxy, amide, cyano, nitrile and/or nitro groups. In a particularly preferred embodiment, R⁷ and/or R⁸ and/or R⁹ are each independently hydrogen, a C₁-C₆-alkyl, C₂-C₆-alkenyl or C₃-C₆-cycloalkyl radical and especially an alkyl radical having 1, 2 or 3 carbon atoms. These radicals may bear up to three substituents. Particularly preferred aliphatic R¹ and/or R² radicals are hydrogen, methyl, ethyl, hydroxyethyl, n-propyl, isopropyl, hydroxypropyl, n-butyl, isobutyl and tert-butyl, hydroxybutyl, n-hexyl, cyclohexyl, n-octyl, n-decyl, n-dodecyl, tridecyl, isotridecyl, tetradecyl, hexadecyl, octadecyl and methylphenyl.

In a further preferred embodiment, R⁷ and R⁸ together with the nitrogen atom to which they are bonded form a ring. This ring has preferably 4 or more than 4, for example 4, 5, 6 or more, ring members. Preferred further ring members are carbon, nitrogen, oxygen and sulfur atoms. The rings may themselves in turn bear substituents, for example alkyl radicals. Suitable ring structures are, for example, morpholinyl, pyrrolidinyl, piperidinyl, imidazolyl and azepanyl radicals.

In a further preferred embodiment, R⁷, R⁸ and/or R⁹ are each independently an optionally substituted C₆-C₁₂-aryl group or an optionally substituted heteroaromatic group having 5 to 12 ring members.

In a further preferred embodiment, R⁷, R⁸ and/or R⁹ are each independently an alkyl radical interrupted by heteroatoms. Particularly preferred heteroatoms are oxygen and nitrogen.

For instance, R⁷, R⁸ and/or R⁹ are each independently preferably radicals of the formula (3)

—(R¹⁰—O)ₐ—R¹¹ (3)

in which
R¹⁰ is an alkylene group having 2 to 6 carbon atoms and preferably having 2 to 4 carbon atoms, for example ethylene, propylene, butylene or mixtures thereof,
R¹¹ is hydrogen, a hydrocarbon radical having 1 to 24 carbon atoms or a group of the formula —R¹⁰—NR¹²R¹³,
a is a number from 2 to 50, preferably from 3 to 25 and especially from 4 to 10 and
R¹², R¹³ are each independently hydrogen, an aliphatic radical having 1 to 24 carbon atoms and preferably 2 to 18 carbon atoms, an aryl group or heteroaryl group having 5 to 12 ring members, a poly(oxyalkylene) group having 1 to 50 poly(oxyalkylene) units, where the polyoxyalkylene units derive from alkylene oxide units having 2 to 6 carbon atoms, or R¹² and R¹³ together with the nitrogen atom to which they are bonded form a ring having 4, 6, 6 or more ring members.

Additionally preferably, R⁷, R⁸ and/or R⁹ are each independently radicals of the formula (4)

—[R¹⁴—N(R¹⁵)]_{b}—(R¹⁵) (4)

in which
R¹⁴ is an alkylene group having 2 to 6 carbon atoms and preferably having 2 to 4 carbon atoms, for example ethylene, propylene or mixtures thereof,
each R¹⁵ is independently hydrogen, an alkyl or hydroxyalkyl radical having up to 24 carbon atoms, for example 2 to 20 carbon atoms, a polyoxyalkylene radical —(R¹⁰—O)ₚ—R¹¹, or a polyiminoalkylene radical —[R¹⁴—N(R¹⁵)]_{q}—(R¹⁵), where R¹⁰, R¹¹, R¹⁴ and R¹⁵ are each as defined above and q and p are each independently 1 to 50 and
b is a number from 1 to 20 and preferably 2 to 10, for example three, four, five or six.

The radicals of the formula (4) contain preferably 1 to 50 and especially 2 to 20 nitrogen atoms.

Particular preference is given to water-soluble alkylamines such as methylamine, dimethylamine, trimethylamine, ethylamine, diethylamine, triethylamine, propylamine and longer-chain mono-, di- and trialkylamines, provided that they are water-soluble. The alkyl chains here may be branched. Equally suitable are oligoamines such as ethylenediamine, diethylenetriamine, triethylenetetramine, tetraethylenepentamine, the higher homologs thereof and mixtures thereof. Further suitable amines in this series are the alkylated, particularly methylated, representatives of these oligoamines, such as N,N-dimethyldiethylenamine, N,N-dimethylpropylamine and longer-chain and/or more highly alkylated amines of the same structure principle. Particularly suitable in accordance with the invention are alkanolamines such as monoethanolamine, diethanolamine, triethanolamine, diglycolamine, triglycolamine and higher homologs, methyldiethanolamine, ethyldiethanolamine, propyldiethanolamine, butyldiethanolamine and longer-chain alkyldiethanolamines, where the alkyl radical may be cyclic and/or branched. Further suitable alkanolamines are dialkylethanolamines such as dimethylethanolamine, diethylethanolamine, dipropylethanolamine, dibutylethanolamine and longer-chain dialkylethanolamines, where the alkyl radical may also be branched or cyclic. In addition, it is also possible in the context of the invention to use aminopropanol, aminobutanol, aminopentanol and higher homologs, and the corresponding mono- and dimethylpropanolamines and longer-chain mono- and dialkylaminoalcohols. Suitable amines are not least specialty amines such as 2-amino-2-methylpropanol (AMP), 2-aminopropanediol, 2-amino-2-ethylpropanediol, 2-aminobutanediol and other 2-aminoalkanols, aminoalkylamine alcohols, tris(hydroxylmethyl)aminomethane, and also end-capped representatives such as methylglycolamine, methyldiglycolamine and higher homologs, di(methylglycol)amine, di(methyldiglycol)amine and higher homologs thereof, and the corresponding Marlines and polyalkylene glycol amines (e.g. Jeffamine®). Particular preference is also given to distillation residues from morpholine synthesis (e.g. AMIX M, CAS No. 68909-77-3). Typically, and in the context of the invention, mixtures of the abovementioned amines are used in order to establish desired pH values.

Further suitable neutralizing agents are the carbonates, hydrogencarbonates, oxides and hydroxides of the alkali metals and/or alkaline earth metals, for example lithium hydroxide, sodium hydroxide, potassium hydroxide, calcium hydroxide, calcium carbonate, calcium hydrogencarbonate and calcium oxide.

The neutralizing agents are used in amounts which are required to establish a pH between 7 and 11. The amounts required for this purpose are preferably, according to the neutralizing agent in the inventive composition, in the range of 1-30%, preferably 5-15%, and in the aqueous metalworking fluid at 0.01-6%, preferably 0.1-1.5% (percent by weight).

The process for preparing pyrrolidonecarboxylic acids of the formula (1) is known, and comprises the reaction of amines of the formula R¹—NH₂ with itaconic acid, and optionally the subsequent neutralizing, as described above.

The water phase of the inventive inverse emulsion may, in a preferred embodiment, comprise various solids, and dissolved singly and multiply charged ions. In a further preferred embodiment, these are doubly or more than doubly charged positive ions. In a preferred embodiment, these are selected from alkaline earth metal ions, especially magnesium and calcium ions, and from ions of diamines or higher amines.

Suitable diamines or higher amines correspond to the formula (2)

NR⁷R⁸R⁹ (2)

in which R⁷, R⁸ and R⁹ are each independently radicals of the formula (4)

—[R¹⁴—N(R¹⁵)]_{b}—(R¹⁵) (4)

in which
R¹⁴ is an alkylene group having 2 to 6 carbon atoms and preferably having 2 to 4 carbon atoms, for example ethylene, propylene or mixtures thereof,
each R¹⁵ is independently hydrogen, an alkyl or hydroxyalkyl radical having up to 24 carbon atoms, for example 2 to 20 carbon atoms, a polyoxyalkylene radical —(R¹⁰—O)ₚ—R¹¹, or a polyiminoalkylene radical —[R¹⁴—N(R¹⁵)]_{q}—(R¹⁵), where R¹⁴ and R¹⁵ are each as defined above and
q and p are each independently 1 to 50, and
R¹⁰ is an alkylene group having 2 to 6 carbon atoms and preferably having 2 to 4 carbon atoms, for example ethylene, propylene, butylene or mixtures thereof,
R¹¹ is hydrogen, a hydrocarbon radical having 1 to 24 carbon atoms or a group of the formula —R¹⁰—NR¹²R¹³,
b is a number from 1 to 20 and preferably 2 to 10, for example three, four, five or six.

The radicals of the formula (4) contain preferably 1 to 50 and especially 2 to 20 nitrogen atoms.

Particular preference is given to oligoamines such as ethylenediamine, diethylenetriamine, triethylenetetramine, tetraethylenepentamine, the higher homologs thereof and mixtures thereof. Further suitable amines in this series are the alkylated, particularly methylated, representatives of these oligoamines, such as N,N-dimethyldiethylenamine, N,N-dimethylpropylamine and longer-chain and/or more highly alkylated amines of the same structure principle.

Other suitable amines are, for example, 1,3-propanediamine, 1,2-propanediamine, neopentanediamine, hexamethylenediamine, octamethylenediamine, isophoronediamine, 4,4′-diaminodicyclohexylmethane, 3,3′-dimethyl-4,4′-diaminodicylohexylmethane, 4,4′-diaminodiphertylmethane, 4,9-dioxadodecane-1,12-diamine, 4,7,10-trioxamidecane-1,13-diamine, 3-(methylamino)propylamine, 3-(cyclohexylamino)propylamine, 2-(diethylamino)ethylamine, 3-(dimethylamino)propylamine, 3-(diethylamino)propylamine, N,N,N′,N′-tetramethyl-1,3-propanediamine, N,N-diethyl-N′,N′-dimethyl-1,3-propanediamine, diethylenetriamine, (3-(2-aminoethyl)aminopropylamine), dipropylenetriamine, N,N-bis-(3-aminopropyl)methylamine, (N,N′-bis(3-aminopropyl)ethylenediamine).

Suitable polyetheramines are, for example, Polyetheramine D 230, Polyetheramine D 400, Polyetheramine D 2000, Polytetrahydrofuranamine 1700, Polyetheramine T 403, Polyetheramine T 5000.

Also suitable are bis(3-dimethylaminopropyl)amine, 2,2′-dimorpholinodiethyl ether, triethylenediamine, triethylenediamine, triethylenediamine, N,N′-dimethylpiperazine, bis(2-dimethylaminoethyl)ether, bis(2-dimethylaminoethyl)ether, pentamethyldiethylenetriamine, N,N,N′-trimethylaminoethylethanolamine, N,N,N′,N′-tetramethyl-1,6-hexanediamine, 1,3,5-tris(dimethylaminopropyl)-sym-hexahydrotriazine, 1,8-diazabicyclo[5.4.0]undec-7-ene, N-(3-aminopropyl)imidazole, 1,2-dimethylimidazole, 1-methylimidazole.

In a further embodiment of the invention, the inventive emulsifiers are formulated in a suitable solvent which is an oleophilic liquid in order to improve the handling thereof at low ambient temperatures below 0° C., for example down to −40° C.

The oleophilic liquid is preferably a material selected from the group consisting of diesel oil, mineral oil, synthetic oils, esters, ethers, acetals, e.g. Flostafluid® 4120 (Clariant), dialkyl carbonates, hydrocarbons and combinations thereof. Preference is given to using environmentally compatible solvents. Particular preference is given to solvents which achieve particularly positive results in ecotoxieity tests for registration in environmentally sensitive regions.

Preferred oleophilic liquids are paraffins, n-paraffins, isoparaffins, for example Isopar® M, dearomatized mineral oil fractions, for example Exxsol® D 100 S, aliphatic alcohols, for example isooctanol, tridecanol, aliphatic esters, ketones, for example diisobutyl ketone, glycols and polyglycols, for example based on ethylene glycol, propylene glycol and butylene glycol, and α-olefins. Preference is given to the formulation of the emulsifiers in the base oil of the inventive composition, i.e. the hydrophobic liquid a).

In a preferred embodiment, in the process for producing the inventive invert emulsions, compounds containing singly or multiply charged ions of the above-described type are added to the water. The amount of such compounds is, based on the weight of the water, 0.1 to 10% by weight, preferably 1 to 5% by weight.

The continuous phase of the inventive inverse emulsion is a hydrophobic liquid. A suitable hydrophobic liquid is any which can be used, for example, for textile, leather and metal treatment, foods, cosmetics, pharmaceuticals, coating materials, in agrochemicals, polymerization, in cleaning and polishing, and in ore extraction and natural gas and mineral oil production.

Particularly preferred inverse emulsions are invert emulsion muds in mineral oil production.

To sink boreholes in rock and bring up the drilling material detached, liquid mud systems based on water and oil are used. Oil-based drilling muds are used in particular in offshore boreholes in the form of what are called invert emulsion muds, in which fine solids (drilling material) are present in W/O emulsions finely dispersed in the continuous oil phase.

In order to bring about the preferred use properties in the overall system described, a multitude of different additives, for example emulsifiers/emulsifier systems, fluid loss additives, viscosity regulators, alkali reserves and weighting agents, are needed. In this respect, reference is made, for example, to the publication by P. A. Boyd et al., “New base oil used in low toxicity oil muds” Journal of Petroleum Technology, 1985, 137-142.

In order to use such invert drilling mud systems practically, the rheological properties thereof must remain relatively constant within a temperature range, i.e. uncontrolled thickening and hence increasing viscosity of the drilling mud solution must be prevented. If the drillpipe gets stuck during operation (called “stuck pipe”; cf. Manual of Drilling fluids Technology, The Netherlands, Baroid/NL Inc., 1985, “Stuck Pipe” chapter), it can be released again only by tire-consuming and costly measures.

In practical application, suitable thinners are therefore added to the drilling mud systems before and during drilling, preferably anionic surfactants from the group of the fatty alcohol sulfates, fatty alcohol ether sulfates and alkylbenzenesulfonates. Although such compounds can effectively control the rheology of the overall system, there is a rise in viscosity at low temperatures of 10° C. and colder, which can then only be controlled with difficulty, if at all.

Suitable borehole treatment compositions should, however, not have any influence on the rheology of the overall system even at elevated temperatures, as can occur during drilling at great depths. The ambient conditions in the case of soil drilling, for example high pressure and pH changes as a result of oxidic gases, also make high demands on the selection of possible components and additives.

Due to the scarcity of fossil resources, ever more boreholes are being sunk in ecologically protected areas. High demands are therefore being made on suitable borehole treatment compositions for reasons of environmental protection in on- and offshore boreholes in respective biodegradability and the toxicity of these substances.

In order to use aqueous drilling mud systems in emulsion form, the additional use of emulsifiers is absolutely necessary. With regard to the chemical nature especially of nonionic emulsifiers, there exists extensive prior art, for example SHINODA et al., Encyclopedia of Emulsion Technology, 1983, vol. 1, 337 to 367; G. L. HOLLIS, Surfactants Europa, third edition, Royal Society of Chemistry, chapter 4, 139-317; M. J. SCHICK, Nonionic Surfactants, Marcel Dekker, INC., New York, 1967; H. W. STACHE, Anionic Surfactants, Marcel Dekker, INC. New York, Basle, Hong Kong; Dr, N. SCHOENFELDT, Grenzflächenaktive Ethylenoxid-Addukte [Interface-active Ethylene Oxide Adducts], Wissenschaftfliche Verlagsanstalt mbH, Stuttgart, 1976.

U.S. Pat. No. 2,908,711 and U.S. Pat. No. 3,035,907 describe oil-soluble reaction products of amines or diamines and itaconic acid, which can be used as antirust additives in fuels or mineral oils.

U.S. Pat. No. 3,218,264 discloses oil-soluble pyrrolidonecarboxylic acid amine salts and uses thereof as corrosion inhibitors in lubricating oils and greases. The amines used for salt formation are oil-soluble.

U.S. Pat. No. 3,224,968 likewise describes oil-soluble amine salts of pyrrolidonecarboxylic acids which use find use as antirust additives in lubricant oils. Again, oil-soluble amines (preferably C₁₂-C₂₀-alkyl-substituted) are used for amine salt formation. U.S. Pat. No. 3,224,975 describes the free pyrrolidonecarboxylic acids for the same use.

GB-A-1 323 061 discloses pyrrolidone derivatives and the use thereof in functional fluids, for example hydraulic fluids. The compounds used have C₁-C₅-alkyl substituents or C₅-C₁₀-aryl substituents on the pyrrolidone nitrogen. In hydraulic fluids, the compounds exhibit anticorrosive properties, even in combination with aliphatic amines.

None of the publications cited describes N-substituted 5-oxopyrrolidin-3-carboxylic acids of the formula (1) as additives for drilling mud solutions.

For the invert emulsion muds with a continuous hydrocarbon phase, for example, fractions of crude oil such as diesel oil, cleaned diesel oil with aromatics content below 0.5% by weight (clean oil), white oils, or conversion products such as olefins, for example α-olefins, polyolefins or alkylbenzenes, are used as a constituent of the continuous phase. These are pure hydrocarbons which are not degraded under the anaerobic conditions in the drilling material sludge on the seabed. For the continuous phase of invert emulsion muds, alcohols, acetals, esters, ethers and triglycerides are also options.

The invert emulsion muds contain reagents which have to ensure the oil wetting of all solids in the mud and of the drilling material drilled out. The drilling material separated out above ground is oil-wetted and often has to be disposed of separately. Offshore there are considerable environmentally damaging effects when the drilling material or mud volumes get into the sea. Drilling material sludge and the heavy mud fall to the seabed, and some flows with the tides and oceanic flows as far as the coasts, for example the mudflats. On this route or its area of spread, the sludge kills all life on the seabed by hydrophobization. Diesel oil was originally the basis of invert emulsion muds. Recently, less toxic diesel oils which have been cleaned to a greater degree have been used, which contain less than 0.5% aromatics and white oils, olefins, polyolefins, various mineral oils with low aromatic content, n-paraffins, isoparaffins and, alkylbenzenes.

Suitable continuous phases in the inventive invert emulsions are, for example, acetals.

Suitable acetals are acetals based on monofunctional aldehydes having 1 to 25, especially 1 to 10, carbon atoms, and monohydric alcohols having 1 to 25, especially 4 to 20 carbon atoms. They may be branched or unbranched, saturated or unsaturated, and aliphatic or aromatic. The acetals may also consist of a mixture which has been prepared from different or from single-chain alcohols and/or aldehydes. In addition, it is also possible to use acetals prepared from dialdehydes, especially having 2 to 10 carbon atoms, such as glyoxal, tartaraldehyde, succinaldehyde, malealdehyde and fumaraldehyde, but preferably glyoxal, with the alcohols mentioned.

The preparation of the acetals is described in EP-A-0 512 501,

Suitable alcohols are linear alcohols, branched alcohols, unsaturated alcohols and/or branched unsaturated alcohols. Preference is given to alcohols having 8 to 25, more preferably 10 to 16, carbon atoms. Especially preferred are linear alcohols having 10 to 16 carbon atoms. The alcohols are preferably oleophilic. Suitable alcohols are especially decanol, dodecanol, tetradecanol, coconut fatty alcohol, lauryl alcohol and α-methyldecanol. The alcohols are available as commercial products.

Suitable continuous phases in the inventive invert emulsions are also oleophilic esters. Suitable oleophilic esters are esters based on mono-, di- and/or trifunctional alcohols and C₁-C₂₅-carboxylic acids.

The monofunctional alcohols are preferably alcohols which have 8 to 25 carbon atoms and may be linear, branched, unsaturated and/or aromatic.

The difunctional alcohols are alcohols having up to 18 carbon atoms, preferably 2 to 18 carbon atoms, which are optionally also present as polyglycol ethers having up to 6 ethylene- and/or propylene alkyls. Examples of difunctional alcohols are ethylene glycol, propylene glycol and butylene glycol, and dialkanolamines such as diethanolamine.

The trifunctional alcohols are alcohols having up to 6 carbon atoms, preferably 2 to 6 carbon atoms, for example glycerol, and trialkanolamines, for example triethanolamine.

The aforementioned C₁-C₂₅-carboxylic acids include mono-, di- and/or trifunctional carboxylic acids which are linear, branched, unsaturated and aromatic.

Examples of monofunctional carboxylic acids of natural origin are coconut fatty acid, lauric acid, myristic acid, palmitic acid, palmitoleic acid, stearic acid, oleic acid, elaidic acid, petroselinic acid, ricinoleic acid, eleostearic acid, linoleic acid, linolenic acid, eicosanoic acid, gadoleic acid, docosanoic acid, erucic acid, tall oil fatty acid and tallow fatty acid.

Examples of difunctional carboxylic acids are oxalic acid, malonic acid, succinic acid and phthalic acid.

An example of a trifunctional carboxylic acid is citric acid.

Suitable continuous phases are also natural oils, i.e. triglycerides of fatty acids. Suitable fatty acids comprise 12 to 22 carbon atoms, for example lauric acid, myristic acid, palmitic acid, palmitoleic acid, stearic acid, oleic acid, elaidic acid, petroselinic acid, ricinoleic acid, eleostearic acid, linoleic acid, linolenic acid, eicosanoic acid, gadoleic acid, docosanoic acid or crude acid. Mixtures with particularly advantageous properties are those which contain principally, i.e. to an extent of at least 50% by weight, glyceryl esters of fatty acids having 16 to 22 carbon atoms and 1, 2 or 3 double bonds.

Examples of suitable oils are rapeseed oil, coriander oil, soya bean oil, cottonseed oil, sunflower oil, castor oil, olive oil, peanut oil, corn oil, almond oil, palm kernel oil, coconut oil, mustardseed oil, bovine tallow and fish oils. Further examples include oils of wheat, jute, sesame, rhea tree nut, arachis oil and linseed oil.

Suitable continuous phases are also oleophilic ethers. Suitable ethers are aliphatic, saturated, or mono- and diunsaturated ethers. The alcohols from which the ethers are formed have in the range from 4 to 36, preferably 6-24, especially 8-18, carbon atoms. It is possible to use ethers formed from one alcohol and mixed ethers formed from two alcohols.

The oleophilic esters and ethers listed are compounds which are frequently obtainable as commercial products. All esters not of natural origin can be prepared by acidic catalysis from the corresponding alcohols and carboxylic acids. Ethers are obtained, for example, by the acidic condensation of alcohols.

The term “oleophilic” herein represents substances whose water solubility at room temperature is below 1% by weight and especially not more than 0.5% by weight.

For the oleophilic phase of an emulsion or invert emulsion for use as a drilling mud, very particular requirements are made on the viscosity and pour point thereof. The properties must enable good pumping under practical conditions, which means that the plastic viscosity of the formulated mud under standard conditions (20° C.) should be not more than 50-100 cP, preferably less than 80 cP.

The viscosity of the oleophilic phase should therefore not exceed 10 cP, but a maximum of 25 cP, at 20° C., and the pour point should be at least below −10° C. Only thus is it possible to formulate a pumpable mud under offshore conditions, for example in the North Sea after shutdowns. In the case of drilling in tropical regions, the viscosities may be somewhat higher, e.g. 15-30 cP, and the pour point may be up to +10° C.

The said formers of oleophilic phases are also suitable formulation ingredients or solvents for the inventive emulsifiers.

The inventive invert emulsions typically comprise

from 20 to 90% by weight of the hydrophobic liquid which forms the continuous phase,
from 5 to 70% by weight of water, and
from 0.5 to 20% by weight of the compound of the formula (1).

When the inventive invert emulsions are used as drilling mud, they may comprise further additives.

Customary additives to water-based O/W emulsion muds are emulsifiers, fluid loss additives, soluble and/or insoluble substances which build up structure viscosity, alkali reserves, inhibitors of unwanted water exchange between drilled formations—for example water-swellable clays and/or salt layers—and the water-based drilling mud, wetting agents for better attachment of the emulsified oil phase on solid surfaces, for example to improve the lubricity, but also to improve the oleophilic occlusion of exposed rock formations or rock faces, disinfectants, for example to inhibit bacterial infestation of such O/W emulsions, and the like. Reference should be made here to the details in the relevant prior art, for example George R. Gray, O. C. H. Darley, “Composition and Properties of Oil Well Drilling Fluids” 4^{th} edition 1980/81, Gulf Publishing Company, Houston, chapter 11, “Drilling Fluid Components”.

It is customary to use finely dispersed additives to increase the mud density. Barium sulfate (barite) is widely used, but calcium carbonate (calcite) or the mixed carbonate of calcium and magnesium (dolomite) are also used.

It is also customarily to use agents to build up the structural viscosity, which at the same time also act as fluid loss additives. Mention should be made here primarily of bentonite, which, is used in an unmodified form in water-based muds and is thus not of ecological concern; in oil-based emulsion muds, organically modified bentonite is also commonly used. For salt water muds, other comparable clays, especially attapulgite and sepiolite, are also of significance in practice. The additional use of organic polymer compounds of natural and/or synthetic origin is also possible. Mention should be made here especially of starch or chemically modified starches, cellulose derivatives such as carboxymethylcellulose, guar gum, xanthan gum, or else purely synthetic water-soluble and/or water-dispersable polymer compounds, especially of the high molecular weight polyacrylamide compound type with or without anionic or cationic modification.

It is also customary to use thinners to regulate the viscosity. The thinners are, for example, tannins and/or quebracho extract. Further examples thereof are lignite and lignite derivatives, especially lignosulfonates. In a preferred embodiment of the invention, the additional use of toxic components is dispensed with, which include here primarily the corresponding salts with toxic heavy metals, such as chromium and/or copper. One example of inorganic thinners is phosphate compounds.

It is also customary to use additives which inhibit unwanted water exchange with, for example, clays. Options here include the additives known from the prior art for water-based drilling muds. These are especially halides and/or carbonates of the alkali metals and/or alkaline earth metals, and corresponding potassium salts may be of particular significance, optionally in combination with lime. Reference is made, for example, to the corresponding publications in “Petroleum Engineer International”, September 1987, 32-40 and “World Oil”, November 1983, 98-97.

It is also customary to use alkali reserves. Options here include inorganic and/or organic bases matched to the overall characteristics of the mud, especially appropriate basic salts or hydroxides of alkali metals and/or alkaline earth metals, and organic bases.

In the case of organic bases, a distinction has to be made between water-soluble organic bases—for example compounds of the diethanolamine type—and virtually water-insoluble bases of markedly oleophilic character. Oleophilic bases of this kind, which are notable especially for at least one relatively long hydrocarbyl radical having, for example, 8 to 36 carbon atoms, are not dissolved in the aqueous phase but in the disperse oil phase. Here, these basic components are of significance in many ways. They can firstly act directly as alkali reserves. They secondly impart a certain positive charge state to the dispersed oil droplet and hence lead to increased interaction with negative surface charges, as encountered especially in the case of hydrophilic clays and those capable of ionic exchange. According to the invention, it is thus possible to influence hydrolytic cleavage and oleophilic occlusion of water-reactive rock layers.

The amount of the assistants and additives used in each case in principle varies within the customary range and can thus be inferred from the relevant literature cited.

The inventive invert emulsion is produced by combining the emulsifier with the oily fluid and the non-oily fluid in a suitable vessel. The fluid is then stirred vigorously or shear-comminuted such that the two liquids are mixed thoroughly. Thereafter, a visual assessment determines whether an emulsion has been formed. An emulsion is considered to be stable when the oily and non-oily liquids essentially do not separate after stirring. In this case, the emulsion remains stable for more than 1 minute after stoppage of the stirring or shearing motion which formed the emulsion. A test for whether an invert emulsion has formed or not is to take a small amount of the emulsion and add it to a vessel containing the oily liquid. If an invert emulsion is present, the emulsion droplet is dispersed in the oily fluid. An alternative test is to measure the electrical stability of the resulting emulsion using a conventionally obtainable emulsion stability test apparatus. In general, in such tests, the voltage applied between the electrodes is increased until the emulsion splits and a current pulse flows between the two electrodes. The voltage required to split the emulsion is considered to be a measure of the stability of the emulsion. Such tests of emulsion stability are known to those skilled in the art and are described on page 166 of the book COMPOSITION AND PROPERTIES OF DRILLING AND COMPLETION FLUIDS, 5^{th} edition, H. C. H. Darley and George R. Gray, Gulf Publishing Company, 1988.

Depending on their use, the inventive invert emulsions may comprise additional chemicals. For example, it is possible to add wetting agents, organophilic clay types, viscosity regulators, weighting agents, bridging agents and fluid loss regulators to the inventive invert emulsions for additional functional properties.

Wetting agents which may be suitable for use in this invention include commercially available crude tall oil, oxidized crude tall oil, surface-active compounds, organic phosphate esters, modified imidazolines and amido amines, alkylaromatic sulfates and sulfonates and the like, and combinations and derivatives thereof. The examples which follow show that inventive emulsifiers are compatible with the abovementioned wetting agents and the inventive invert emulsions are not adversely affected. They can be partly or else completely replaced by the inventive emulsifiers.

Organophilic clay types, preferably amine-treated clay types, may be useful as viscosity regulators in the fluid compositions of the present invention. Other viscosity regulators, such as oil-soluble polymers, polyamide resins, polycarboxylic acids and soaps, can likewise be used. The amount of the viscosity regulator used in the composition may vary depending on the alternate use of the composition. However, a range of about 0.1 to 6% by weight is sufficient for most applications. The compounds mentioned are known to those skilled in the art and can be purchased commercially.

Suspension media suitable for use in this invention include organophilic clay types, amine-treated clay types, oil-soluble polymers, polyamide resins, polycarboxylic acids and soaps. The amount of the viscosity regulator used in the composition may vary depending on the ultimate use of the composition. However, a range of about 0.1 to 6% by weight is sufficient for most applications. These media too are commercially available.

Weighting agents suitable for use in this invention include, for example, hematite, magnetite, iron oxides, illmenite, barite, siderite, celestine, dolomite and calcite, or chalk. The amount of such added materials depends on the desired density of the ultimate composition. Typically, a weighting material is added in order that a drilling mud density of up to about 2.88 kg/l (24 pounds per gallon) is obtained. The weighting material is preferably added up to 2.52 kg/l (21 pounds per gallon) and more preferably up to 2.34 kg/l (19.5 pounds per gallon).

Fluid loss regulators, also known as fluid loss additives, typically act by coating the walls of the borehole when the borehole is bored. Suitable fluid loss regulators which can be used in this invention include modified brown coal types, asphalt compounds, gilsonite, organophilic humates which are prepared by reacting huminic acid with amides or polyalkylene polyamines, for example organophilic leonardite, and other nontoxic fluid loss additives. Typically, the fluid loss additives are added in amounts of less than 10 and preferably less than about 5% by weight of the fluid.

### General Information Relevant for the Examples

These tests were conducted according to the methods in API Bulletin RP 13B-2, 1990. The abbreviations which, follow are sometimes used in describing the test results.

“PV” is the plastic viscosity measured in the unit centipoise [cP], which is used in the calculation of the viscosity properties of a drilling mud.

“AV” is the apparent viscosity measured in the unit centipoise [cP] is used for the calculation of the rheological properties of a drilling mud.

“YP” is the yield point measured in pounds per 100 square feet [lb/100 ft², 1 lb/100 ft²=0.049 kgm⁻²], which is used in the calculation of the rheology properties of a drilling mud.

The “gel strength” is a measure in pounds per 100 square feet [lb/100 ft², 1 lb/100 ft²=0.049 kgm⁻²] for the suspension properties or the thixotropic properties of a drilling fluid.

“HTHP” is the high-temperature high-pressure liquid loss of the drilling mud measured in milliliters [ml/30 min] according to API-Bulletin RP 13 B-2, 1990.

The pressure differential applied is typically measured in pounds per square inch [psi, 1 psi=6.895·10⁻² bar]. Accordingly, 500 psi=34.475 bar.

One American pound (lb) corresponds to 0.4536 kg.

One US gallon corresponds to 3.785 L.

### EXAMPLES

For all examples adduced, the following equipment was used:

Fann® Hamilton Beach Laboratory Mixer 3 Speed Model N 5009 and accompanying stirrer cup, set to level 2

### Fann® Electrical Stability Tester Model 23D

Fann® Model 35SA Rheometer in the customary R1-B1-F1 configuration.

This means that rotor 1, bob 1 and spring 1 were used.

Baroid® Testing Equipment HTHP liquid loss test apparatus complete with nitrogen supply and 500 ml high-pressure test cells and aging cells

### Baroid® Testing Equipment Roller Oven Model 77

All equipment and procedures used correspond to API Recommended Practice 13 B-2 (oil-based muds). The person skilled in the art is aware of technical terms and abbreviations.

The examples which follow are intended to illustrate the invention; advantages over the prior art are to be shown. Emphasis lies principally on performance as an emulsifier. In no way are the illustrative laboratory muds to be considered as fully developed field muds.

For examples 1 to 32, the following test sequence was selected:

### Formulation of the Emulsion Mud

The following components were combined and mixed in a stirrer cup of a Hamilton Beach mixer in the sequence specified below:

1. Oleophilic phase
2. Primary emulsifier→stirring time 1 min.
3. time→stirring time 1 min.
4. Fluid loss additive→stirring time 1 min.
5. Secondary emulsifier→stirring time 1 min.
6. Saturated CaCl₂ solution→stirring time 10 min,
7. Organophilic bentonite→stirring time 15 min.
then the electrical stability was determined
8. Barium sulfate→stirring time 10 min.
then the electrical stability was determined again.

### Performance Testing:

All muds were performance tested in the sequence below. The results are summarized in tabular form.
1. Determination of the emulsion stability before and after the addition of barium sulfate.
2. Determination of the rheological data with FANN 35 at 65° C. (150° F.).
3. Gel strength after 10 seconds and after 10 minutes at 65° C. (150° F.)
4. Dynamic aging of the mud at 65° C. (150° F.) for 16 hours in a roller oven.
5. Determination of the emulsion stability after aging.
6. Measurement of the rheological data after aging with FANN 35 at 65° C. (150° F.).
7. Determination of the gel strength with FANN 35 after 10 seconds and after 10 minutes at 65° C. (150° F.).
8. Determination of the HTHP fluid loss at 150° C. (approx. 300° F.), a pressure differential of 500 psi in 30 minutes.

### Example 1

### Comparative

According to the procedure described above, 170 ml of diesel (diesel #2), 6 g of commercial emulsifier mixture of oxidized tall oil and fatty acid amido amine, 3 g of lime, 6 g of commercial fluid loss additive (organophilic leonardite), 3 g of commercial carboxylic acid-capped fatty acid polyamide and 75 ml of saturated CaCl₂ solution and 2 g of organophilic bentonite are mixed. After the determination of the emulsion stability, 470 g of barium sulfate were incorporated and the electrical stability was determined again. The commercial carboxylic acid-capped fatty acid polyamide is the reaction product of tall oil fatty acid with a polyamine mixture which comprises principally triamines and tetramines, and which has been crosslinked subsequently with citric acid.

In all tables below, the emulsion stability is reported as electrical stability in volts. In all tables below, the rheology figures reported are readout values of the FANN 35 viscosimeter, the configuration thereof being bob 1, rotor 1 and spring 1.

### Emulsion Stability

| | |
|---|---|
| Electrical stability before barium sulfate [V] | 300 |
| Electrical stability after barium sulfate [V] | 250 |
| Electrical stability after aging [V] | 330 |

### Rheology at 65° C. (150° F.)

| Measurement parameter | | Before aging | After aging |
|---|---|---|---|
| 600 rpm | | 161 | 150 |
| 300 rpm | | 118 | 108 |
| 200 rpm | | 102 | 91 |
| 100 rpm | | 81 | 71 |
| 6 rpm | | 48 | 38 |
| 3 rpm | | 47 | 32 |
| 10 second gel strength | [lb/100 ft²] | 47 | 32 |
| 10 minute gel strength | [lb/100 ft²] | 53 | 34 |
| Apparent viscosity μa | [cP] | 81 | 75 |
| Plastic viscosity μp | [cP] | 43 | 42 |
| Yield point Y.P. | [lb/100 ft²] | 75 | 66 |
| HTHP Fluid Loss @ 500 | [ml/30 min.] | — | 12.4 |
| psi, 149° C. (300° F.) | | | |

Example 1 shows, as prior art, the properties of a highly weighted invert emulsion mud under laboratory conditions. For this high-solids laboratory mud, quite a high rheology is normal and should be taken into account in comparison with the further examples. The HTHP fluid loss at 12.4 ml/130 min is quite high even for a laboratory mud. Values less than 10 ml/30 min are desirable.

### Example 2

### N-Oleyl(pyrrolidin-2-one)-4-carboxylic acid as primary emulsifier

According to the procedure described above, 170 ml of diesel (diesel #2), 6 g of N-oleyl(pyrrolidin-2-one)-4-carboxylic acid, 3 g of lime, 6 g of commercial fluid loss additive (organophilic leonardite), 3 g of commercial carboxylic acid-capped fatty acid polyimide and 75 ml of saturated CaCl₂ solution, 2 g of organophilic bentonite were mixed. After the determination of the emulsion stability, 470 g of barium sulfate were incorporated and the electrical stability was determined again.

### Emulsion Stability

| | |
|---|---|
| Electrical stability before barium sulfate [V] | 530 |
| Electrical stability after barium sulfate [V] | 850 |
| Electrical stability after aging [V] | 920 |

### Rheology at 65° C. (150° F.)

| Measurement parameter | | Before aging | After aging |
|---|---|---|---|
| 600 rpm | | 129 | 143 |
| 300 rpm | | 89 | 102 |
| 200 rpm | | 72 | 85 |
| 100 rpm | | 54 | 65 |
| 6 rpm | | 24 | 29 |
| 3 rpm | | 22 | 26 |
| 10 second gel strength | [lb/100 ft² | 22 | 27 |
| | lb/100 ft²] | | |
| 10 minute gel strength | [lb/100 ft² | 24 | 29 |
| | lb/100 ft²] | | |
| Apparent viscosity μa | [cP] | 65 | 72 |
| Plastic viscosity μp | [cP] | 40 | 41 |
| Yield point Y.P. | [lb/100 ft²] | 49 | 61 |
| HTHP Fluid Loss @ 500 | [ml/30 min.] | — | 8.4 |
| psi, 149° C. (300° F.) | | | |

A very high electrical stability is found, higher than in example 1. The rheological properties are likewise better than in example 1. The apparent viscosity, the plastic viscosity and the gel strengths are lower than in example 1. The liquid loss at 8.4 ml is likewise much lower than in example 1. The filtrate is free of water.

### Example 3

### N-Octadecyl(pyrrolidine-2-one)-4-carboxylic acid as primary emulsifier

According to the procedure described above, 170 ml of diesel (diesel #2), 6 g of N-octadecyl(pyrrolidin-2-one)-4-carboxylic acid, 3 g of lime, 6 g of commercial fluid loss additive (organophilic leonardite), 3 g of commercial carboxylic acid-capped fatty acid polyamide and 75 ml of saturated CaCl₂ solution, 2 g of organophilic bentonite were mixed. After the determination of the emulsion stability, 470 g of barium sulfate were incorporated and the electrical stability was determined again.

### Emulsion Stability

| | |
|---|---|
| Electrical stability before barium sulfate [V] | 410 |
| Electrical stability after barium sulfate [V] | 430 |
| Electrical stability after aging [V] | 500 |

### Rheology at 65° C. (150° F.)

| Measurement parameter | | Before aging | After aging |
|---|---|---|---|
| 600 rpm | | 143 | 154 |
| 300 rpm | | 101 | 111 |
| 200 rpm | | 84 | 93 |
| 100 rpm | | 64 | 72 |
| 6 rpm | | 33 | 38 |
| 3 rpm | | 29 | 33 |
| 10 second gel strength | [lb/100 ft²] | 30 | 33 |
| 10 minute gel strength | [lb/100 ft²] | 34 | 38 |
| Apparent viscosity μa | [cP] | 72 | 77 |
| Plastic viscosity μp | [cP] | 42 | 43 |
| Yield point Y.P. | [lb/100 ft²] | 59 | 68 |
| HTHP Fluid Loss @ 500 | [ml/30 min.] | — | 4.0 |
| psi, 149° C. (300° F.) | | | |

### Examples 4-7

### N-Oleyl(pyrrolidine-2-one)-4-carboxylic acid partial salts as primary emulsifier

In example
4 by A: N-oleyl(pyrrolidine-2-one)-4-carboxylic acid/polypropylene glycol diamine salt with a mean molecular weight of 230 g/mol (Jeffamin® D 230 from Huntsman) in a molar ratio of 2:1
5 by B: N-oleyl(pyrrolidine-2-one)-4-carboxylic acid/ethanol amine salt in a molar ratio of 1:1
6 by C: N-oleyl(pyrrolidine-2-one)-4-carboxylic acid/polypropylene glycol-co-ethylene glycol monoamine salt with a mean molecular weight of 1000 g/mol (Jeffamin® M 1000 from Huntsman) in a molar ratio of 1:1
7 by D: N-oleyl(pyrroildine-2-one)-4-carboxylic acid/morpholine distillation residue salt (AMIX M from BASF) in a molar ratio of 1:1

According to the procedure described above, 170 ml of diesel (diesel #2), 6 g of N-oleyl(pyrrolidine-2-one)-4-carboxylic acid salt A, B, C and D, 3 g of lime, 6 g of commercial fluid loss additive (organophilic leonardite), 3 g of commercial carboxylic acid-capped fatty acid polyamide and 75 ml of saturated CaCl₂ solution, 2 g of organophilic bentonite were mixed. After the determination of the emulsion stability, 470 g of barium sulfate were incorporated and the electrical stability was determined again.

### Emulsion Stability

| Example | 4 | 5 | 6 | 7 |
|---|---|---|---|---|
| Electrical stability before barium sulfate [V] | 330 | 380 | 380 | 500 |
| Electrical stability after barium sulfate [V] | 480 | 560 | 260 | 710 |
| Electrical stability after aging [V] | 420 | 530 | 400 | 980 |

### Rheology at 65° C. (150° F.)

| | Before aging - example | | | | After aging - example | | | |
|---|---|---|---|---|---|---|---|---|
| Measurement parameter | 4 | 5 | 6 | 7 | 4 | 5 | 6 | 7 |
| 600 rpm | 120 | 127 | 149 | 115 | 127 | 135 | 152 | 123 |
| 300 rpm | 80 | 87 | 100 | 78 | 87 | 94 | 106 | 84 |
| 200 rpm | 65 | 72 | 82 | 63 | 71 | 79 | 88 | 69 |
| 100 rpm | 48 | 54 | 61 | 47 | 53 | 60 | 67 | 52 |
| 6 rpm | 23 | 25 | 29 | 22 | 26 | 28 | 33 | 25 |
| 3 rpm | 21 | 23 | 27 | 20 | 24 | 26 | 29 | 23 |
| 10 second gel strength | 21 | 23 | 27 | 20 | 24 | 26 | 29 | 23 |
| [lb/100 ft²] | | | | | | | | |
| 10 minute gel strength | 24 | 26 | 32 | 23 | 27 | 28 | 32 | 25 |
| [lb/100 ft²] | | | | | | | | |
| Apparent viscosity μa [cP] | 60 | 64 | 75 | 58 | 64 | 69 | 76 | 62 |
| Plastic viscosity μp [cP] | 40 | 40 | 49 | 37 | 40 | 41 | 46 | 39 |
| Yield point Y.P. [lb/100 ft²] | 40 | 47 | 51 | 41 | 47 | 53 | 80 | 45 |
| HTHP Fluid loss @ 500 psi, | — | — | — | — | 6.8 | 5.2 | 5.8 | 6.4 |
| 149° C. (300° F.) [ml/30 min.] | | | | | | | | |

### Example 8

### N-Oleyl(pyrrolidin 2-one)-4-carboxylic acid as secondary emulsifier

According to the procedure described above, 170 ml of diesel (diesel #2), 6 g of commercial emulsifier mixture of oxidized tall oil and fatty acid amido amine, 3 g of lime, 6 g of commercial fluid loss additive (organophilic leonardite), 3 g of N-oleyl(pyrrolidin-2-one)-4-carboxylic acid and 75 ml of saturated CaCl₂ solution, 2 g of organophilic bentonite were mixed. After the determination of the emulsion stability, 470 g of barium sulfate were incorporated and the electrical stability was determined again.

### Emulsion Stability

| | |
|---|---|
| Electrical stability before barium sulfate [V] | 530 |
| Electrical stability after barium sulfate [V] | 850 |
| Electrical stability after aging [V] | 920 |

### Rheology at 65° C. (150° F.)

| Measurement parameter | | Before aging | After aging |
|---|---|---|---|
| 600 rpm | | 140 | 152 |
| 300 rpm | | 99 | 109 |
| 200 rpm | | 82 | 90 |
| 100 rpm | | 62 | 68 |
| 6 rpm | | 30 | 32 |
| 3 rpm | | 27 | 28 |
| 10 second gel strength | [lb/100 ft²] | 27 | 28 |
| 10 minute gel strength | [lb/100 ft²] | 30 | 31 |
| Apparent viscosity μa | [cP] | 70 | 76 |
| Plastic viscosity μp | [cP] | 41 | 43 |
| Yield point Y.P. | [lb/100 ft²] | 58 | 76 |
| HTHP Fluid Loss @ 500 | [ml/30 min.] | — | 8.8 |
| psi, 149° C. (300° F.) | | | |

### Example 9

### N-Octadecyl(pyrrolidin-2-one)-4-carboxylic acid as secondary emulsifier

According to the procedure described above, 170 ml of diesel (diesel #2), 6 g of commercial emulsifier mixture of oxidized tail oil and fatty acid amide amine, 3 g of lime, 6 g of commercial fluid loss additive (organophilic leonardite), 3 g of N-octadecyl(pyrrolidin-2-one)-4-carboxylic acid and 75 ml of saturated CaCl₂ solution, 2 g of organophilic bentonite were mixed. After the determination of the emulsion stability, 470 g of barium sulfate were incorporated and the electrical stability was determined again.

### Emulsion Stability

| | |
|---|---|
| Electrical stability before barium sulfate [V] | 410 |
| Electrical stability after barium sulfate [V] | 530 |
| Electrical stability after aging [V] | 500 |

### Rheology at 65° C. (150° F.)

| Measurement parameter | | Before aging | After aging |
|---|---|---|---|
| 600 rpm | | 137 | 155 |
| 300 rpm | | 96 | 111 |
| 200 rpm | | 81 | 93 |
| 100 rpm | | 62 | 71 |
| 6 rpm | | 29 | 33 |
| 3 rpm | | 27 | 30 |
| 10 second gel strength | [lb/100 ft²] | 27 | 29 |
| 10 minute gel strength | [lb/100 ft²] | 29 | 33 |
| Apparent viscosity μa | [cP] | 69 | 78 |
| Plastic viscosity μp | [cP] | 41 | 44 |
| Yield point Y.P. | [lb/100 ft²] | 55 | 67 |
| HTHP Fluid Loss @ 500 | [ml/30 min.] | — | 10.0 |
| psi, 149° C. (300° F.) | | | |

### Examples 10-13

### N-Oleyl(pyrrolidin-2-one)-4-carboxylic acid partial salts as secondary emulsifiers

In example
10 by A: N-oleyl(pyrrolidine-2-one)-4-carboxylic acid/polypropylene glycol diamine salt with a mean molecular weight of about 230 g/mol (Jeffamin® D 230 from Huntsman) in a molar ratio of 2:1
11 by B: N-oleyl(pyrrolidine-2-one)-4-carboxylic acid/ethanol amine salt in a molar ratio of 1:1
12 by C: N-oleyl(pyrrolidine-2-one)-4-carboxylic acid/polypropylene glycol-co-ethylene glycol monoamine salt with a mean molecular weight of 1000 g/mol (Jeffamin® M 1000 from Huntsman) in a molar ratio of 1:1
13 by D: I\1-oleyl(pyrrolidine-2-one)-4-carboxylic acid/morpholine distillation residue salt (ADMIX M from BASF) in a molar ratio of 1:1

According to the procedure described above, 170 ml of diesel (diesel #2), 6 g of commercial emulsifier mixture of oxidized tall oil and fatty acid amido amine, 3 g of lime, 6 g of commercial fluid loss additive (organophilic leonardite), 3 g of N-oleyl(pyrrolidin-2-one)-4-carboxylic acid salt A, B, C or D and 75 ml of saturated CaCl₂ solution, 2 g of organophilic bentonite were mixed. After the determination of the emulsion stability, 470 g of barium sulfate were incorporated and the electrical stability was determined again.

### Emulsion Stability

| Example | 10 | 11 | 12 | 13 |
|---|---|---|---|---|
| Electrical stability before barium sulfate [V] | 440 | 450 | 420 | 430 |
| Electrical stability after barium sulfate [V] | 750 | 740 | 710 | 750 |
| Electrical stability after aging [V] | 800 | 840 | 750 | 770 |

### Rheology at 65° C. (150° F.)

| | Before aging - example | | | | After aging - example | | | |
|---|---|---|---|---|---|---|---|---|
| Measurement parameter | 10 | 11 | 12 | 13 | 10 | 11 | 12 | 13 |
| 600 rpm | 130 | 121 | 127 | 115 | 144 | 139 | 134 | 137 |
| 300 rpm | 90 | 81 | 87 | 78 | 102 | 97 | 93 | 95 |
| 200 rpm | 74 | 65 | 72 | 63 | 85 | 8β | 78 | 80 |
| 100 rpm | 56 | 48 | 55 | 46 | 64 | 60 | 59 | 60 |
| 6 rpm | 26 | 22 | 26 | 22 | 30 | 29 | 29 | 29 |
| 3 rpm | 24 | 20 | 24 | 20 | 27 | 26 | 26 | 26 |
| 10 second gel strength | 24 | 20 | 24 | 20 | 27 | 26 | 26 | 26 |
| [lb/100 ft²] | | | | | | | | |
| 10 minute gel strength | 26 | 24 | 28 | 23 | 30 | 29 | 29 | 28 |
| [lb/100 ft²] | | | | | | | | |
| Apparent viscosity μa [cP] | 65 | 61 | 64 | 58 | 72 | 70 | 67 | 69 |
| Plastic viscosity μp [cP] | 40 | 40 | 40 | 37 | 42 | 42 | 41 | 42 |
| Yield point Y.P. [lb/100 ft²] | 50 | 41 | 47 | 41 | 60 | 55 | 52 | 53 |
| HTHP Fluid Loss @ 500 psi, | — | — | — | — | 9.2 | 9.0 | 9.2 | 8.4 |
| 149° C. (300° F.) [ml/30 min.] | | | | | | | | |

### Example 14

### N-Oleyl(pyrrolidin-2-one)-4-carboxylic, acid/morpholine distillation residue salt (AMIX M from BASF) in a molar ratio of 1:1 as primary and secondary emulsifier

According to the procedure described above, 170 ml of diesel (diesel #2), 9 g of N-oleyl(pyrrolidin-2-one)-4-carboxylic acid morpholine distillation residue (AMIX M from BASF) in a molar ratio of 1:1, 3 g of lime, 6 g of commercial fluid loss additive and 75 ml of saturated CaCl₂ solution, 2 g of organophilic bentonite were mixed. After the determination of the emulsion stability, 470 g of barium sulfate were incorporated and the electrical stability was determined again.

### Emulsion Stability

| | |
|---|---|
| Electrical stability before barium sulfate [V] | 510 |
| Electrical stability after barium sulfate [V] | 700 |
| Electrical stability after aging [V] | 870 |

### Rheology at 65° C. (150° F.)

| Measurement parameter | | Before aging | After aging |
|---|---|---|---|
| 600 rpm | | 114 | 129 |
| 300 rpm | | 75 | 91 |
| 200 rpm | | 62 | 74 |
| 100 rpm | | 45 | 55 |
| 6 rpm | | 21 | 27 |
| 3 rpm | | 19 | 25 |
| 10 second gel strength | [lb/100 ft²] | 19 | 25 |
| 10 minute gel strength | [lb/100 ft²] | 21 | 27 |
| Apparent viscosity μa | [cP] | 57 | 65 |
| Plastic viscosity μp | [cP] | 39 | 38 |
| Yield point Y.P. | [lb/100 ft²] | 36 | 53 |
| HTHP Fluid Loss @ 500 | [ml/30 min.] | — | 6.0 |
| psi, 149° C. (300° F.) | | | |

### Example 15

### N-Oleyl(pyrrolidin-2-one)-4-carboxylic acid/ethanolamine salt in a molar ratio of 1:1 as primary and secondary emulsifier

According to the procedure described above, 170 ml of diesel (diesel #2), 9 g of N-oleyl(pyrrolidin-2-one)-4-carboxylic acid morpholine distillation residue (AMIX M from BASF) in a molar ratio of 1:1, 3 g of lime, 6 g of commercial fluid loss additive (organophilic leonardite) and 75 ml of saturated CaCl₂ solution. 2 g of organophilic bentonite were mixed. After the determination of the emulsion stability, 470 g of barium sulfate were incorporated and the electrical stability was determined again.

### Emulsion Stability

| | |
|---|---|
| Electrical stability before barium sulfate [V] | 450 |
| Electrical stability after barium sulfate [V] | 860 |
| Electrical stability after aging [V] | 970 |

### Rheology at 65° C. (150° F.)

| Measurement parameter | | Before aging | After aging |
|---|---|---|---|
| 600 rpm | | 126 | 132 |
| 300 rpm | | 86 | 94 |
| 200 rpm | | 70 | 79 |
| 100 rpm | | 53 | 60 |
| 6 rpm | | 24 | 28 |
| 3 rpm | | 21 | 25 |
| 10 second gel strength | [lb/100 ft²] | 21 | 25 |
| 10 minute gel strength | [lb/100 ft²] | 24 | 27 |
| Apparent viscosity μa | [cP] | 63 | 66 |
| Plastic viscosity μp | [cP] | 40 | 38 |
| Yield point Y.P. | [lb/100 ft²] | 46 | 56 |
| HTHP Fluid Loss @ 500 | [ml/30 min.] | — | 6.0 |
| psi, 149° C. (300° F.) | | | |

### Example 16

### Lauryl polypropylene oxide N-(pyrrolidin-2-one)-4-carboxylic acid with 2 to 3 propylene oxide units (n=2 to 3) as primary and secondary emulsifier

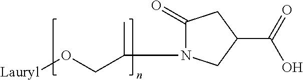

According to the procedure described above, 170 ml of diesel (diesel #2), 5 g of lauryl polypropylene oxide N-(pyrrolidin-2-one)-4-carboxylic acid, 3 g of lime, 6 g of commercial fluid loss additive and 75 ml of saturated CaCl₂ solution, 2 g of organophilic bentonite were mixed. After the determination of the emulsion stability, 470 g of barium sulfate were incorporated and the electrical stability was determined again.

### Emulsion Stability

| | |
|---|---|
| Electrical stability before barium sulfate [V] | 440 |
| Electrical stability after barium sulfate [V] | 680 |
| Electrical stability after aging [V] | 780 |

### Rheology at 65° C. (150° F.)

| Measurement parameter | | Before aging | After aging |
|---|---|---|---|
| 600 rpm | | 117 | 119 |
| 300 rpm | | 79 | 84 |
| 200 rpm | | 64 | 66 |
| 100 rpm | | 46 | 48 |
| 6 rpm | | 19 | 20 |
| 3 rpm | | 17 | 18 |
| 10 second gel strength | [lb/100 ft²] | 17 | 18 |
| 10 minute gel strength | [lb/100 ft²] | 19 | 19 |
| Apparent viscosity μa | [cP] | 59 | 60 |
| Plastic viscosity μp | [cP] | 38 | 35 |
| Yield point Y.P. | [lb/100 ft²] | 41 | 49 |
| HTHP Fluid Loss @ 500 | [ml/30 min.] | — | 5.8 |
| psi, 149° C. (300° F.) | | | |

In example 16, in a departure from the previous examples, only 5 g of emulsifier were used. In spite of this, excellent rheological values were found. The HTHP fluid loss was also very good at 5.8 ml. The inventive emulsifier can thus be used more sparingly than the prior art here only 55.6%. Nevertheless, better performance results were achieved.

### Examples 17-20

### The Replacement of the Oleophilic Phase, Diesel #2, with N-Paraffin and α-Olefin as Base Oil with Simultaneous Use of a Single Inventive Emulsifier

In example
17 n-paraffin and N-oleyl(pyrrolidine-2-one)-4-carboxylic acid/morpholine distillation residue salt in a molar ratio of 1:1 (AMIX M from BASF) were used
18 α-olefin and 1′-oleyl(pyrrolidine-2-one)-4-carboxylic acid/morpholine distillation residue salt in a molar ratio of 1:1 (AMIX M from BASF) were used
19 n-paraffin and N-oleyl(pyrrolidine-2-one)-4-carboxylic acid/ethanolamine salt in a molar ratio of 1:1 were used
20 α-olefin and NI-oleyl(pyrrolidine-2-one)-4-carboxylic acid/ethanolamine salt in a molar ratio of 1:1 were used

According to the procedure described above, 170 ml of the particular base oil, 9 g of the inventive emulsifier, 3 g of lime, 6 g of commercial fluid loss additive (organophilic leonardite) and 75 ml of saturated CaCl₂ solution, 2 g of organophilic bentonite were mixed. After the determination of the emulsion stability, 470 g of barium sulfate were incorporated and the electrical stability was determined again.

### Emulsion Stability

| Example | 17 | 18 | 19 | 20 |
|---|---|---|---|---|
| Electrical stability before barium sulfate [V] | 270 | 350 | 270 | 1040 |
| Electrical stability after barium sulfate [V] | 470 | 750 | 550 | 720 |
| Electrical stability after aging [V] | 610 | 920 | 580 | 600 |

### Rheology at 65° C. (150° F.)

| | Before aging - example | | | | After aging - example | | | |
|---|---|---|---|---|---|---|---|---|
| Measurement parameter | 17 | 18 | 19 | 20 | 17 | 18 | 19 | 20 |
| 600 rpm | 75 | 54 | 86 | 65 | 71 | 57 | 92 | 64 |
| 300 rpm | 41 | 32 | 48 | 39 | 39 | 35 | 51 | 38 |
| 200 rpm | 29 | 25 | 35 | 30 | 28 | 27 | 37 | 29 |
| 100 rpm | 17 | 17 | 21 | 29 | 17 | 19 | 23 | 20 |
| 6 rpm | 4 | 5 | 6 | 7 | 5 | 7 | 7 | 8 |
| 3 rpm | 3 | 4 | 5 | 6 | 4 | 5 | 6 | 6 |
| 10 second gel strength | 4 | 5 | 7 | 7 | 5 | 6 | 7 | 7 |
| [lb/100 ft²] | | | | | | | | |
| 10 minute gel strength | 7 | 7 | 9 | 13 | 8 | 8 | 10 | 8 |
| [lb/100 ft²] | | | | | | | | |
| Apparent viscosity μa [cP] | 38 | 27 | 43 | 33 | 36 | 29 | 46 | 32 |
| Plastic viscosity μp [cP] | 34 | 22 | 38 | 26 | 32 | 22 | 41 | 26 |
| Yield point Y.P. [lb/100 ft²] | 7 | 10 | 10 | 13 | 7 | 13 | 10 | 12 |
| HTHP Fluid Loss @ 500 psi, | — | — | — | — | 3.6 | 5.2 | 4.4 | 6.4 |
| 149° C. (300° F.) [ml/30 min.] | | | | | | | | |

No settling of the barite was observed.

All examples based on inventive emulsifiers exhibited excellent results in diesel-based muds. The inventive emulsifiers were compatible with existing mud components. It was possible to exchange primary, secondary and also both emulsifier systems. Thus, using inventive emulsifiers, a simple mud system was obtained. The rheology and fluid loss were in some cases considerably improved compared to the prior art. In example 16 it was shown that a reduced proportion of inventive emulsifier also gives excellent performance results.

Typically, the exchange of the base oil requires adjustment or complete restructuring of the mud system. In examples 17 to 20, the diesel base oil in a standard mud optimized for diesel oil was replaced by more environmentally friendly n-paraffin or α-olefin. Remarkably, the mud is still exceptionally stable. The rheology is in some cases much lower. It is at the limit of serviceability, but barite precipitation was not observed to any significant degree.

### Examples 21 to 32

### Exchange of the Diesel Base Oil for n-Paraffin and α-Olefin with Simultaneous Use of a Single Inventive Emulsifier

In example
21 n-paraffin and N-oleyl(pyrrolidin-2-one)-4-carboxylic acid morpholine distillation residue salt in a molar ratio of 1:1 (AMIX M from BASF) were used,
22 α-olefin and N-oleyl(pyrrolidin-2-one)-4-carboxylic acid morpholine distillation residue salt in a molar ratio of 1:1 (AMIX M from BASF) were used,
23 n-paraffin and N-oleyl(pyrrolidin-2-one)-4-carboxylic acid ethanolamine salt in a molar ratio of 1:1 were used,
24 α-olefin and N-oleyl(pyrrolidin-2-one)-4-carboxylic acid ethanolamine salt in a molar ratio of 1:1 were used,
25 n-paraffin and N-oleyl(pyrrolidin-2-one)-4-carboxylic acid polypropylene glycol diamine salt with a mean molecular weight of 230 g/mol (Jeffamine® D230 from Huntsman) in a molar ratio of approx. 2:1 were used,
26 α-olefin and N-oleyl(pyrrolidin-2-one)-4-carboxylic acid polypropylene glycol diamine salt with a mean molecular weight of approx. 230 g/mol (Jeffamine® D230 from Huntsman) in a molar ratio of approx. 2:1 were used,
27 n-paraffin and N-oleyl(pyrrolidin-2-one)-4-carboxylic acid were used,
28 α-olefin and N-oleyl(pyrrolidin-2-one)-4-carboxylic acid were used,
29 n-paraffin and N-oleyl(pyrrolidin-2-one)-4-carboxylic acid polypropylene glycol diamine salt with a mean molecular weight of approx. 430 g/mol (Jeffamine® D400 from Huntsman) in a molar ratio of approx, 2:1 were used,
30 α-olefin and N-oleyl(pyrrolidin-2-one)-4-carboxylic acid polypropylene glycol diamine salt with a mean molecular weight of approx. 430 g/mol (Jeffamine® D400 from Huntsman) in a molar ratio of approx. 2:1 were used,
31 n-paraffin and N-oleyl(pyrrolidin-2-one)-4-carboxylic acid polyalkylene glycol diamine salt, based predominantly on a PEG backbone, with a mean molecular weight of approx. 600 g/mol (Jeffamine® ED600 from Huntsman) in a molar ratio of approx. 2:1 were used,
32 α-olefin and N-oleyl(pyrrolidin-2-one)-4-carboxylic acid polypropylene glycol diamine salt, based predominantly on a PEG backbone, with a mean molecular weight of approx. 600 mmol (Jeffamine® ED600 from Huntsman) in a molar ratio of approx. 2:1 were used.

According to the procedure described above, 170 ml of the particular base oil, 9 g of the inventive emulsifier, 3 g of lime, 6 g of commercial fluid loss additive (organophilic leonardite) and 75 ml of saturated CaCl₂ solution, 2 g of organophilic bentonite were mixed. After the determination of the emulsion stability, 470 g of barium sulfate were incorporated and the electrical stability was determined again.

### Emulsion Stability

| Example | 21 | 22 | 23 | 24 |
|---|---|---|---|---|
| Electrical stability before barium sulfate [V] | 350 | 400 | 420 | 410 |
| Electrical stability after barium sulfate [V] | 280 | 590 | 520 | 780 |
| Electrical stability after aging [V] | 760 | 800 | 580 | 500 |

### Rheology at 65° C.

| | Before aging - example | | | | After aging - example | | | |
|---|---|---|---|---|---|---|---|---|
| Measurement parameter | 21 | 22 | 23 | 24 | 21 | 22 | 23 | 24 |
| 600 rpm | 80 | 67 | 80 | 74 | 65 | 72 | 88 | 65 |
| 300 rpm | 45 | 40 | 45 | 44 | 37 | 45 | 49 | 73 |
| 200 rpm | 33 | 32 | 32 | 34 | 29 | 36 | 35 | 45 |
| 100 rpm | 20 | 22 | 20 | 23 | 19 | 25 | 21 | 34 |
| 6 rpm | 6 | 8 | 5 | 9 | 6 | 10 | 6 | 23 |
| 3 rpm | 4 | 7 | 4 | 8 | 5 | 9 | 5 | 9 |
| 10 second gel strength | 4 | 7 | 5 | 8 | 5 | 10 | 7 | 8 |
| [lb/100 ft²] | | | | | | | | |
| 10 minute gel strength | 6 | 10 | 10 | 11 | 6 | 11 | 11 | 10 |
| [lb/100 ft²] | | | | | | | | |
| Apparent viscosity μa [cP] | 40 | 34 | 40 | 37 | 33 | 36 | 44 | 13 |
| Plastic viscosity μp [cP] | 35 | 27 | 35 | 30 | 28 | 27 | 39 | 37 |
| Yield point Y.P. [lb/100 ft²] | 10 | 13 | 10 | 14 | 9 | 18 | 10 | 28 |
| HTHP Fluid Loss @ 500 psi, | — | — | — | — | 6.4 | 6.4 | 3.2 | 6.0 |
| 149° C. (300° F.) [ml/30 min.] | | | | | | | | |

In some cases the rheology is quite low, but no settling of the barite was observed.

### Emulsion Stability

| Example | 25 | 26 | 27 | 28 |
|---|---|---|---|---|
| Electrical stability before barium sulfate [V] | 430 | 400 | 280 | 510 |
| Electrical stability after barium sulfate [V] | 660 | 530 | 270 | 750 |
| Electrical stability after aging [V] | 570 | 550 | 420 | 560 |

### Rheology at 65° C.

| | Before aging - example | | | | After aging - example | | | |
|---|---|---|---|---|---|---|---|---|
| Measurement parameter | 25 | 26 | 27 | 28 | 25 | 26 | 27 | 28 |
| 600 rpm | 56 | 69 | 65 | 57 | 49 | 72 | 53 | 86 |
| 300 rpm | 29 | 45 | 34 | 33 | 26 | 48 | 27 | 36 |
| 200 rpm | 21 | 35 | 24 | 25 | 19 | 38 | 19 | 26 |
| 100 rpm | 13 | 25 | 15 | 17 | 12 | 27 | 12 | 16 |
| 6 rpm | 3 | 9 | 4 | 5 | 3 | 11 | 3 | 4 |
| 3 rpm | 2 | 7 | 3 | 4 | 3 | 10 | 2 | 3 |
| 10 second gel strength | 3 | 8 | 4 | 5 | 4 | 10 | 3 | 3 |
| [lb/100 ft²] | | | | | | | | |
| 10 minute gel strength | 4 | 10 | 5 | 7 | 5 | 11 | 5 | 8 |
| [lb/100 ft²] | | | | | | | | |
| Apparent viscosity μa [cP] | 28 | 35 | 33 | 29 | 25 | 36 | 27 | 33 |
| Plastic viscosity μp [cP] | 27 | 24 | 31 | 24 | 23 | 24 | 26 | 30 |
| Yield point Y.P. [lb/100 ft²] | 2 | 21 | 3 | 9 | 3 | 24 | 1 | 6 |
| HTHP Fluid Loss @ 500 psi, | — | — | — | — | 10.2 | 6.4 | 10.1 | 5.2 |
| 149° C. (300° F.) [ml/30 min.] | | | | | | | | |

No settling of the barite was observed.

### Examples 33 to 37

### Exchange of the #2 Diesel Base Oil for Isobutyraldehyde 2-Ethylhexyl Acetal (Hostafluid® 4120) with Simultaneous Use of a Single Inventive Emulsifier

In example
33 isobutyraldehyde 2-ethylhexyl acetal (Hostafluid® 4120) and N-oleyl (pyrrolidin-2-one)-4-carboxylic acid morpholine distillation residue salt in a molar ratio of 1:1 (AMIX M from BASF) were used,
34 isobutyraldehyde 2-ethylhexyl acetal (Hostafluid® 4120) and N-oleyl (pyrrolidin-2-one)-4-carboxylic acid ethanolamine salt in a molar ratio of 1:1 were used,
35 isobutyraldehyde 2-ethylhexyl acetal (Hostafluid® 4120) and N-oleyl (pyrrolidin-2-one)-4-carboxylic acid polypropylene glycol diamine salt with a mean molecular weight of 230 g/mol (Jeffamine® D230 from Huntsman) in a molar ratio of approx. 21 were used,
36 isobutyraldehyde 2-ethylhexyl acetal (Hostafluid® 4120) and N-oleyl (pyrrolidin-2-one)-4-carboxylic acid were used,
37 isobutyraldehyde 2-ethylhexyl acetal (Hostafluid® 4120) and N-oleyl (pyrrolidin-2-one)-4-carboxylic acid polypropylene glycol diamine salt with a mean molecular weight of 600 g/mol (Jeffamine® ED600 from Huntsman) in a molar ratio of approx. 2:1 were used.

According to the procedure described above, 178 ml of Hastafluid® 4120, 9 g of the inventive emulsifier, 3 g of lime, 6 g of commercial fluid loss additive (organophilic leonardite) and 75 ml of saturated CaCl₂ solution, 2 g of organophilic bentonite were mixed. After the determination of the emulsion stability, 470 g of barium sulfate were incorporated and the electrical stability was determined again.

### Emulsion Stability

| Example | 29 | 30 | 31 | 32 |
|---|---|---|---|---|
| Electrical stability before barium sulfate [V] | 550 | 410 | 730 | 560 |
| Electrical stability after barium sulfate [V] | 420 | 750 | 850 | 450 |
| Electrical stability after aging [V] | 880 | 820 | 300 | 850 |

### Rheology at 65° C.

| | Before aging - example | | | | After aging - example | | | |
|---|---|---|---|---|---|---|---|---|
| Measurement parameter | 29 | 30 | 31 | 32 | 29 | 30 | 31 | 32 |
| 600 rpm | 170 | 144 | 186 | 219 | 196 | 177 | 155 | 260 |
| 300 rpm | 94 | 80 | 102 | 126 | 111 | 99 | 85 | 152 |
| 200 rpm | 68 | 56 | 73 | 91 | 80 | 71 | 61 | 111 |
| 100 rpm | 39 | 32 | 42 | 54 | 47 | 41 | 35 | 66 |
| 6 rpm | 7 | 6 | 7 | 10 | 10 | 9 | 6 | 14 |
| 3 rpm | 5 | 5 | 5 | 8 | 8 | 7 | 4 | 11 |
| 10 second gel strength | 6 | 7 | 5 | 9 | 9 | 8 | 4 | 12 |
| [lb/100 ft²] | | | | | | | | |
| 10 minute gel strength | 9 | 9 | 8 | 20 | 12 | 10 | 7 | 23 |
| [lb/100 ft²] | | | | | | | | |
| Apparent viscosity μa [cP] | 85 | 72 | 93 | 110 | 98 | 89 | 78 | 130 |
| Plastic viscosity μp [cP] | 78 | 64 | 84 | 93 | 85 | 78 | 70 | 108 |
| Yield point Y.P. [lb/100 ft²] | 18 | 16 | 18 | 33 | 26 | 21 | 15 | 44 |
| HTHP Fluid Loss @ 500 psi, | — | — | — | — | 3.2 | 4.0 | 4.8 | 1.6 |
| 149° C. (300° F.) [ml/30 min.] | | | | | | | | |

### Emulsion Stability

| Example | 33 |
|---|---|
| Electrical stability before barium sulfate [V] | 440 |
| Electrical stability after barium sulfate [V] | 340 |
| Electrical stability after aging [V] | 350 |

### Rheology at 65° C.

| Measurement parameter | | Before aging | After aging |
|---|---|---|---|
| 600 rpm | | 140 | 109 |
| 300 rpm | | 76 | 59 |
| 200 rpm | | 53 | 41 |
| 100 rpm | | 30 | 23 |
| 6 rpm | | 4 | 3 |
| 3 rpm | | 3 | 2 |
| 10 second gel strength | [lb/100 ft²] | 4 | 2 |
| 10 minute gel strength | [lb/100 ft²] | 6 | 5 |
| Apparent viscosity μa | [cP] | 70 | 55 |
| Plastic viscosity μp | [cP] | 64 | 50 |
| Yield point Y.P. | [lb/100 ft²] | 12 | 9 |
| HTHP Fluid Loss @ 500 | [ml/30 min.] | — | 9.2 |
| psi, 300° F. | | | |

### Example 38 (Comparative)

### Prior Art with Commercial Emulsifiers

200 ml of diesel #2 were homogenized in the stir cup with 4 g of organophilic clay and 3 g of lime in a Hamilton-Beach mixer for 15 minutes. Thereafter, 6 g of commercial emulsifier based on a tall oil reaction product and 3 g of oxidized tall oil fatty acid were added and incorporated in the Hamilton-Beach mixer for 5 minutes. 54 ml of saturated CaCl₂ solution were poured gradually into the Hamilton-Beach mixer with high shear and mixed for 10 min. Thereafter, 5 g of gilsonite were incorporated by mixing for 10 minutes. The electrical stability of the mud was determined before the addition of barium sulfate. Then 324 g of barite and 15 g of synthetic fine drilling dust were incorporated by mixing in the Hamilton-Beach mixer for 10 minutes. The electrical stability was tested again, then the rheological values before aging. Dynamic aging was effected in a roller oven at 65° C. for 16 hours.

### Emulsion Stability

| Comparative example | 34 |
|---|---|
| Electrical stability before barium sulfate [V] | 320 |
| Electrical stability after barium sulfate [V] | 140 |
| Electrical stability after aging [V] | 170 |

### Rheology at 65° C.

| Measurement parameter | | Before aging | After aging |
|---|---|---|---|
| 600 rpm | | 91 | 80 |
| 300 rpm | | 66 | 57 |
| 200 rpm | | 56 | 47 |
| 100 rpm | | 44 | 36 |
| 6 rpm | | 26 | 20 |
| 3 rpm | | 24 | 19 |
| 10 second gel strength | [lb/100 ft²] | 24 | 21 |
| 10 minute gel strength | [lb/100 ft²] | 30 | 25 |
| Apparent viscosity μa | [cP] | 46 | 40 |
| Plastic viscosity μp | [cP] | 25 | 23 |
| Yield point Y.P. | [lb/100 ft²] | 41 | 34 |
| HTHP Fluid Loss @ 500 | [ml/30 min.] | — | 10.0 |
| psi, 150° C. | | | |

### Examples 39 and 40

For the two examples which follow, 200 ml of diesel #2 were homogenized in the stir cup with 4 g of organophilic clay and 3 g of lime in a Hamilton-Beach mixer for 15 minutes. Thereafter, 6 g of commercial emulsifier based on a tall oil reaction product and 6 g of an inventive emulsifier formulation were added and incorporated in the Hamilton-Beach mixer for 5 minutes. 54 ml of saturated CaCl₂ solution were poured gradually into the Hamilton-Beach mixer with high shear and mixed for 10 min. Thereafter, 5 g of gilsonite were incorporated by mixing for 10 minutes. The electrical stability of the mud was determined before the addition of barium sulfate. Then 324 g of barite and 15 g of synthetic fine drilling dust were incorporated by mixing in the Hamilton-Beach mixer for 10 minutes. The electrical stability was tested again, then the rheological values before aging. Dynamic aging was effected in each case at 65° C. for 16 hours.

For example 39, an inventive emulsifier formulation of 50% N-olely(pyrrolidin-2-one)-4-carboxylic acid morpholine distillation residue salt in a molar ratio of 1:1 (AMIX M from BASF) in isooctanol was used.

For example 40, an inventive emulsifier formulation of 50% N-olely(pyrrolidin-2-one)-4-carboxylic acid monoethanolamine salt in a molar ratio of 1:1 in isooctanol was used.

### Emulsion Stability

| Example | 39 | 40 |
|---|---|---|
| Electrical stability before barium sulfate [V] | 470 | 390 |
| Electrical stability after barium sulfate [V] | 250 | 110 |
| Electrical stability after aging [V] | 280 | 300 |

### Rheology at 65° C.

| | Before aging - | | After aging - | |
|---|---|---|---|---|
| | example | | example | |
| Measurement parameter | 39 | 40 | 39 | 40 |
| 600 rpm | 77 | 73 | 70 | 69 |
| 300 rpm | 53 | 49 | 47 | 46 |
| 200 rpm | 43 | 40 | 33 | 38 |
| 100 rpm | 33 | 31 | 28 | 28 |
| 6 rpm | 17 | 16 | 14 | 14 |
| 3 rpm | 15 | 14 | 13 | 13 |
| 10 second gel strength [lb/100 ft²] | 16 | 15 | 14 | 14 |
| 10 minute gel strength [lb/100 ft²] | 21 | 20 | 18 | 17 |
| Apparent viscosity μa [cP] | 39 | 37 | 35 | 35 |
| Plastic viscosity μp [cP] | 24 | 24 | 23 | 23 |
| Yieid point Y.P. [lb/100 ft²] | 29 | 25 | 24 | 23 |
| HTHP Fluid Loss @ 500 psi, | — | — | 3.4 | 3.8 |
| 149° C. (300° F.) [ml/30 min.] | | | | |

In examples 39 and 40 too, an improved electrical stability and HTHP fluid loss are found compared to comparative example 38, with comparable rheology.

### Example 41

Ecotoxicology data were obtained for two compounds. Although the acute toxicity was comparable to other emulsifiers, the example compounds exhibited a slight tendency to bioaccumulation. The limit for registration in Norway under HOCNF for the distribution between water and octanol, log p_{o/w}, is 3. We achieved 1.6 for N-oleyl (pyrrolidin-2-one)-4-carboxylic acid morpholine distillation residue salt in a molar ratio of approx. 1:1 (e.g. AMIX M from BASF) and 1.1 for N-oleyl(pyrrolidin-2-one)-4-carboxylic acid monoethanolamine salt in a molar ratio of approx. 1:1. The biodegradability according to OECD 306 test after 28 days for our two example compounds N-oleyl(pyrrolidin-2-one)-4-carboxylic acid morpholine distillation residue salt in a molar ratio of approx. 1:1 (e.g. AMIX M from BASF) and N-oleyl(pyrrolidin-2-one)-4-carboxylic acid monoethanolamine salt in a molar ratio of approx, 1:1 was 70% and 69% respectively, while the tall oil fatty acid amido amine/imidazoline mixture Dodicor® 4605 produced by Clariant exhibited a biodegradability under OECD 306 conditions of only 14.8% in 28 days.

### Example 41

### Ecotoxicology Data

| | OleylPyCOO-AmixM | | | OleylPyCOO-MEA | | |
|---|---|---|---|---|---|---|
| | Protocol ISO 10253: 2008 | | | Protocol ISO 10253: 2006 | | |
| Skelotonema Costatum | Time | (hrs) | 72 | Time | (hrs) | 72 |
| | EC50 | (mg/l) | 1.6 | EC50 | (mg/l) | 0.44 |
| | 72 h EC90 | (mg/l) | 3.0 | 72 h EC90 | (mg/l) | 0.58 |
| | NOEC@72 h | (mg/l) | 0.56 | NOEC @72 h | (mg/l) | 0.32 |
| | Protocol ISO 14669: 1999 | | | Protocol ISO 14669: 1999 | | |
| Artica Tonsa | Time | (hrs) | 48 | Time | (hrs) | 48 |
| | LC50 | (mg/l) | 1.6 | LC50 | (mg/l) | 2.8 |
| | 48 h LC90/100 | (mg/l) | 3.0/9.1 | 48 h LC90/100 | (mg/l) | 4.2/9.0 |
| | 48 h NOEC | (mg/l) | <1.0 | 48 h NOEC | (mg/l) | 0.30 |
| | Protocol OSPAR 2005, Part A | | | Protocol OSPAR 2005, Part A | | |
| Corophium Volulator | Time | (hrs) | 10 | Time | (hrs) | 10 |
| | LC5@10 days | (mg/kg) | 693 | LC5@ 10 days | (mg/kg) | 433 |
| | NOEC @ 10 days | (mg/kg) | 524 | NOEC@ 10 days | (mg/kgl) | 160 |
| Bioaccumulation | Protocol CLA slow stirring | | | Protocol CLA slow stirring | | |
| | log Pow | | 1.6 | log Pow | | 1.1 |
| Biodegradability | Protocol OECD 306 | | | Protocol OECD 306 | | |
| | Time | (days) | 28 | Time | (days) | 28 |
| | Degradation | (%) | 70 | Degradation | (%) | 69 |

OleylPyCOO-AMIX M = inventive compound where R¹ = oleyl, as salt with AMIX M
OleylPyCOO-MEA = inventive compound where R¹ = oleyl, as salt with monoethanolamine

1. An inverse emulsion comprising
a) a hydrophobic liquid as a continuous phase
b) water as a disperse phase, and
c) a compound of the formula (1) 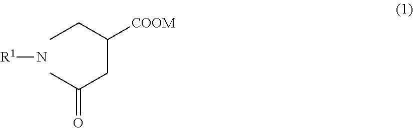
in which
R¹ is a hydrocarbyl group having 6 to 30 carbon atoms or an R⁵—O—X— group
M is hydrogen, alkali metal, alkaline earth metal or an ammonium group
R⁵ is a hydrocarbyl group having 6 to 30 carbon atoms
X is C₂-C₆-alkylene or a poly(oxyalkylene) group of the formula 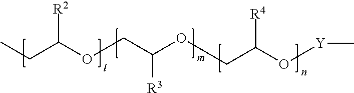
in which
l is a number from 1 to 50,
m, n are independent of l and are each independently a number from 0 to 50,
R², R³, R⁴ are each independently hydrogen, CH₃ or CH₂CH₃
Y is C₂-C₆-alkylene.

2. An inverse emulsion as claimed in claim 1, in which the water phase comprises doubly or more than doubly charged positive ions.

3. An inverse emulsion as claimed in claim 2, in which the doubly or more than doubly charged positive ions are selected from the group consisting of magnesium ions, calcium ions, and ions of diamines or higher amines which correspond to the formula (2)
NR⁷R⁸R⁹ (2)
in which R⁷, R⁸ and R⁹ are each independently a radical of the formula (4)
—[R¹⁴—N(R¹⁵)]_{b}—(R¹⁵) (4)
in which
R¹⁴ is an alkylene group having 2 to 6 carbon atoms or mixtures thereof,
each R¹⁵ is independently hydrogen, an alkyl or hydroxyalkyl radical having up to 24 carbon atoms, a polyoxyalkylene radical —(R¹⁰—O)ₚ—R¹¹ or a polyiminoalkylene radical —[R¹⁴—N(R¹⁵)]_{q}—(R¹⁵) where R¹⁴ and R¹⁵ are each as defined above, and
q and p are each independently from 1 to 50,
R¹⁰ is an alkylene group having 2 to 6 carbon atoms or mixtures thereof,
R¹¹ is hydrogen, a hydrocarbyl radical having 1 to 24 carbon atoms or a group of the formula —R¹⁰—NR¹²R¹³,
b is a number of from 1 to 20.

4. An inverse emulsion as claimed in claim 1, in which R¹ is a linear or branched, aliphatic C₁₂-C₂₄ hydrocarbyl radical having at least one double bond.

5. An inverse emulsion as claimed in claim 1, in which the continuous phase comprises at least one constituent selected from the group consisting of diesel oil, cleaned diesel oil with aromatics content below 0.5% by weight (clean oil), white oils, α-olefins, polyolefins, n-paraffins, isoparaffins, alkylbenzenes, alcohols, acetals, esters, ethers and triglycerides.

6. An inverse emulsion as claimed in claim 1, which comprises 20 to 90% by weight of the hydrophobic liquid a), 5% to 70% by weight of water and 0.5 to 20% by weight of the compound of the formula (1), based on the weight of the inverse emulsion.

7. A process for producing an inverse emulsion, comprising the step of mixing of a hydrophobic liquid, water and a compound of the formula (1) 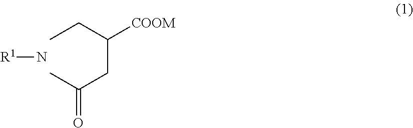
in which
R¹ is a hydrocarbyl group having 6 to 30 carbon atoms or an R⁵—O—X— group
M is hydrogen, alkali metal, alkaline earth metal or an ammonium group
R⁵ is a hydrocarbyl group having 6 to 30 carbon atoms
X is C₂-C₆-alkylene or a poly(oxyalkylene) group of the formula 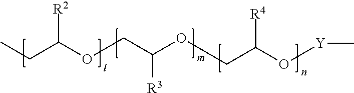
in which
l is a number from 1 to 50,
m, n are independent of l and are each independently a number from 0 to 50,
R², R³, R⁴ are each independently hydrogen, CH₃ or CH₂CH₃
Y is C₂-C₆-alkylene.

8. A process as claimed in claim 7, wherein compounds containing doubly or more than doubly positively charged ions are added to the water phase.

9. A process as claimed in claim 8, in which the doubly or more than doubly charged positive ions are selected from the group consisting of magnesium ions, calcium ions, and ions of diamines or higher amines which correspond to the formula (2)
NR⁷R⁸R⁹ (2)
in which R⁷, R⁸ and R⁹ are each independently a radical of the formula (4)
—[R¹⁴—N(R¹⁵)]_{b}—(R¹⁵) (4)
in which
R¹⁴ is an alkylene group having 2 to 6 carbon atoms or mixtures thereof,
each R¹⁵ is independently hydrogen, an alkyl or hydroxyalkyl radical having up to 24 carbon atoms, a polyoxyalkylene radical —(R¹⁰—O)ₚ—R¹¹ or a polyiminoalkylene radical —[R¹⁴—N(R¹⁵)]_{q}—(R¹⁵) where R¹⁴ and R¹⁵ are each as defined above, and
q and p are each independently from 1 to 50,
R¹⁰ is an alkylene group having 2 to 6 carbon atoms or mixtures thereof,
R¹¹ is hydrogen, a hydrocarbyl radical having 1 to 24 carbon atoms or a group of the formula —R¹⁰—NR¹²R¹³,
b is a number of from 1 to 20.

10. An emulsifier, in an inverse emulsion wherein the inverse emulsion comprises a hydrophobic liquid as a continuous phase and water as a disperse phase, comprising a compound of the formula (1) 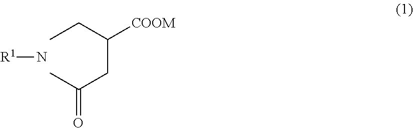
in which
R¹ is a hydrocarbyl group having 6 to 30 carbon atoms or an R⁵—O—X— group
M is hydrogen, alkali metal, alkaline earth metal or an ammonium group
R⁵ is a hydrocarbyl group having 6 to 30 carbon atoms
X is C₂-C₆-alkylene or a poly(oxyalkylene) group of the formula 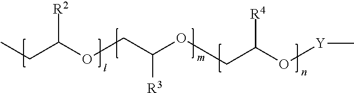
in which
l is a number from 1 to 50,
m, n are independent of l and are each independently a number from 0 to 50,
R², R³, R⁴ are each independently hydrogen, CH₃ or CH₂CH₃
Y is C₂-C₆-alkylene.

11. An invert emulsion drilling fluid comprising the inverse emulsion as claimed in claim 1.

12. A composition comprising 10-90% by weight of at least one compound of the formula (1) 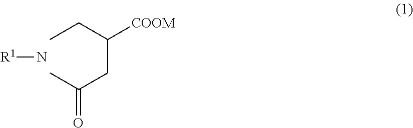
in which
R¹ is a hydrocarbyl group having 6 to 30 carbon atoms or an R⁵—O—X— group
M is hydrogen, alkali metal, alkaline earth metal or an ammonium group
R⁵ is a hydrocarbyl group having 6 to 30 carbon atoms
X is C₂-C₆-alkylene or a poly(oxyalkylene) group of the formula 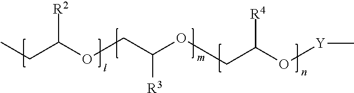
in which
l is a number from 1 to 50,
m, n are independent of l and are each independently a number from 0 to 50,
R², R³, R⁴ are each independently hydrogen, CH₃ or CH₂CH₃
Y is C₂-C₆-alkylene
and an oleophilic liquid selected from the group consisting of diesel oil, cleaned diesel oil with aromatics content below 0.5% by weight (clean oil), white oils, α-olefins, polyolefins, n-paraffins, isoparaffins, alkylbenzenes, alcohols, acetals, esters, ethers and triglycerides.
